(19) United States
(12) Patent Application Publication
(10) Pub. No.: US 2010/0239618 A1
(43) Pub. Date: Sep. 23, 2010

(54) TOOTHPASTE

(75) Inventor: Vaclav Velkoborsky, Zurich (CH)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: SWISSDENT COSMETICS AG, Zurich (CH)

(21) Appl. No.: 12/280,751

(22) PCT Filed: Feb. 28, 2007
(86) PCT No.: PCT/CH07/00103

§ 371 Date: Jun. 9, 2010

(30) Foreign Application Priority Data
Feb. 28, 2006 (CH) 314/06

(51) Int. Cl. A61K 8/03; A61K 8/22; A61K 8/64; A61Q 90/00

(52) U.S. Cl. 424/401; 424/613; 424/94.63; 424/94.65

(57) ABSTRACT

The present invention relates to a toothpaste comprising one or more micronized unencapsulated peroxide compounds with improved properties with regard to cleaning and visual brightening of natural teeth, false teeth and/or polymer fillings of teeth; to its production and use.

The present invention concerns a toothpaste with improved properties regarding cleaning and visual whitening of natural teeth, false teeth, and/or polymer fillings of teeth, and its manufacture and use.

Toothpastes are tooth cleaning agents comprising a composition consisting of (1) abrasive powder, (2) moistening agent, (3) binder, (4) water, (5) flavors, (6) detergent and (7) preservatives in small amounts. In addition, they may contain dyes and other additives that have a positive effect on oral and dental hygiene. They are used first of all for removal of dirt using a toothbrush. However, toothpastes also serve for the removal and prevention of the buildup of plaque and tartar and for the regulation and regeneration of the gum. Therefore toothpastes frequently also contain other components, such as fluorides, vitamins, herb extracts, etc.

A frequently desired cosmetic effect of toothpastes is also the whitening of the surface of teeth, both of natural as well as false teeth, as well as tooth filings and tooth prostheses. Frequently the problem arises here that the transition from natural to artificial tooth substance is not completely smooth, so that the smallest pores and gaps already lead to deposits that have a different color from that of the teeth and are difficult to remove. Such locations lead to the formation of dark edges or spots in time. Peroxides are known as chemical whiteners and are used in toothpastes, too. These are reactive oxidizing agents, which have two main disadvantages: (1) they are toxic, and (2) they liberate oxygen when they come into contact with water, which is a problem in the packaging and storage life of toothpastes. For these reasons, generally a content of about 0.5 to 5% peroxide is customary in toothpastes (U.S. Pat. No. 5,041,280, U.S. Pat. No. 4,603,045, U.S. Pat. No. 4,405,599). US 2002/0064541 describes a formulation containing a peroxide compound in a micro-encapsulated form, whereby the microcapsules are of the “core-shell” type and have a specific shell. Furthermore, the use of such microcapsules in tooth treatment agents has been disclosed. U.S. Pat. No. 5,403,578 describes a toothpaste containing 3.5-11% calcium peroxide in the micro-encapsulated form.

Therefore, the task arises to make a toothpaste available that makes gentle, improved cleaning of natural and false teeth, tooth fillings and tooth prostheses possible with simultaneous whitening of the surfaces, including the edges. Furthermore, the task arises to make a toothpaste available that is inexpensive and can be manufactured from simple components.

One or several of these tasks are solved by the toothpaste according to patent Claim 1. Advantageous embodiments are given in the dependent claims and in the specification. Other aspects of the invention are given in the independent claims and in the specification.

Within the framework of the present invention general, preferred and especially preferred definitions/areas can be combined arbitrarily with one another. Similarly, individual definitions can be omitted.

Accordingly, the invention is concerned in a first aspect with a toothpaste that contains micronized bleaching agents, such as perborates and/or peroxide, preferably peroxide, especially preferably calcium peroxide. As a result, the micronized bleaching agents (“nanoparticles with improved surface activity”) can penetrate into the smallest pores and gaps and can remain active there for a longer time than is the case with non-micronized peroxide of a corresponding concentration. This leads to an improved bleaching effect on the partially irregular surface of the teeth, especially in transitions from natural to artificial tooth material and at the edges. The micronization of the peroxide makes this effect possible even at lower concentrations. In addition to the bleaching effect, the micronized peroxide also generally has a favorable effect on oral hygiene, since it is toxic for bacteria, specifically for anaerobic bacteria. Anaerobic bacteria cause caries and bad breath. They are located not only in the plaque but also in deposits of the pharynx and the oral cavity, especially on the tongue. Moreover, with micronized peroxide an anti-inflammatory action is achieved, which leads specifically to reduced bleeding of the gums.

Furthermore, it was found that the toothpaste according to the invention, which contains unencapsulated micronized peroxide compounds, exhibits surprising stability in the packaging and at the same time exhibits very good action during use.

In an advantageous embodiment, the invention concerns a toothpaste for the cleaning and visual whitening of natural and/or false teeth and tooth fillings, characterized by the fact that it contains micronized peroxide.

In another advantageous embodiment, the invention is concerned with a toothpaste containing one or several micronized, unencapsulated peroxide compounds.

Preferably, after micronization the diameter of about 50% of the particles is less than about 2-3 micrometers, whereby at least 90% of the particles have a diameter of less than about 5 to about 7 micrometers. These particles have all together a larger surface and therefore more active sites, and permit better bleaching of the teeth during application. Without being bound by theory, we start from the assumption that the time of contact of a micronized peroxide particle with the tooth is increased because these nanoparticles can penetrate into the pores and stay there, while the non-micronized peroxide diffuses away from the tooth again rapidly. In a preferred embodiment, the said micronized peroxide compound is produced with a jet mill.

As is clear from the discussion above, the peroxide compound used is unencapsulated.

Within the framework of the present invention, the terms “peroxide” and “peroxide compound” are used synonymously. Generally all peroxide compounds can be used that are known in relation with toothpaste and/or oral hygiene and can be micronized as a solid. The peroxide that is preferred for the invention is calcium peroxide. It is present according to the invention at a concentration above about 0.01%. The lower limit of the peroxide concentration preferably lies at about 0.05%, especially preferably at 0.1%. Normally the peroxide concentration is not higher than about 10%, preferably not higher than 5%, and especially preferably not higher than 2% or the concentration approved for the particular purpose (the Swiss standard for peroxide in cosmetics lies at 0.1%). Percentage is always understood as weight % based on O₂²⁻.

Since many deposits on teeth contain proteins, in another embodiment the invention also provides a toothpaste with micronized peroxide, which additionally contains enzymes with proteolytic activity. Such proteases help in destroying the protein-containing deposits on the teeth, as a result of which better access for the peroxide particles to the tooth surface lying under the deposits is made possible. Similarly, bacterial deposits, also called plaque, are attacked by such proteases because the bacterial surface proteins are substrates for these proteases. In addition, peroxide itself is toxic for many bacteria. Thus there is a synergistic effect when using micronized peroxide, especially calcium peroxide, together with proteases, because better bleaching of the teeth is made possible ough a simultaneous reduction of the bacterial- and protein-containing deposits.

As proteases, in the present invention cysteine proteases are preferred, specifically papain and bromelain. Papain is a sulfhydryl protease with low substrate specificity that is obtained from the latex juice of Carica papaya. Bromelain is a composition that is obtained from pineapples and contains several cysteine proteases. Papain and bromelain may be present individually in the toothpaste. Together and in combination with micronized peroxide, these proteases have an advantageous effect on the bleaching of teeth and therefore in a preferred embodiment of the present invention, all three components are contained in the toothpaste.

The toothpaste of the present invention comprises pastes and gels with a consistency suitable for the cleaning of teeth, and this must be neither too firm nor too fluid. Such a toothpaste contains, in addition to water, at least one abrasive agent, preferably silicic acid (SiO₂), more preferably Sident 8 and Sident 22 S (Degussa AG) in the ratio of 9:5.

In an advantageous embodiment the toothpaste according to the invention contains moistening agents, for example, glycerol, sorbitol, propylene glycol, polyglycols, preferably glycerol and/or sorbitol.

In an advantageous embodiment the toothpaste according to the invention contains binders, for example, gels of plant origin such as cellulose, cellulose ether or pectin, xanthan, carbomers or silicic acids or polysaccharides from algae, known as carrageenan. Preferably carrageenan is used for the present invention.

In an advantageous embodiment, the toothpaste according to the invention contains at least one detergent from the group comprising betains, ether sulfates and sarcosinates. Preferably it is cocamidopropyl-betain, preferably at a concentration of approximately 2%.

Not necessarily but preferably the toothpaste according to the invention also contains one or several components from the group of preservatives, for example methylparaben; flavors and aromas; fluoride-containing compounds, preferably as sodium monofluorophosphate; cosmetic dyes, vitamins, preferably vitamin E; coenzymes, preferably ubiquinone and optionally other additives.

In an advantageous embodiment, the toothpaste according to the invention contains vitamin E and ubiquinone.

In a preferred embodiment, the invention therefore concerns a toothpaste with a pH value in the range from 5 to 8, especially preferably between about 6.5 and about 7.5. It is also desirable that the toothpaste according to the invention have an approximately neutral pH.

In another aspect, the present invention concerns the manufacture of a toothpaste as described above. The manufacture of the toothpaste according to the invention is carried out by combining and mixing the components in a certain sequence. First, the additives, such as enzymes, coenzymes, vitamins, sweeteners and/or fluoride are added individually to a mixture of water, preservative and moistening agent and dissolved in it. Components that are insoluble in water, for example ubiquinone or vitamin E, can hereby be added in the form of liposomes. Then the binder is added, and the mixture obtained is allowed to swell and is homogenized. Then the addition of the abrasive agent and the micronized peroxide follows. Finally, the aromas and colors are incorporated. At the very end the detergent is added and the resulting mixture is homogenized. Therefore, the invention concerns a method for the manufacture of toothpaste comprising the steps i) preparation of a solution of water, moistening agents and preservatives and soluble additives of the group of enzymes, coenzymes, vitamins, sweeteners, fluoride-containing compounds, whereby insoluble compounds can be added in the form of liposomes; ii) addition of binder with subsequent swelling and homogenization; iii) addition of abrasive agent and micronized peroxide compound and homogenization; iv) addition of aroma and color substances and homogenization.

In another aspect, the present invention concerns the use of a toothpaste for the cleaning and optical whitening of natural and/or false teeth and tooth fillings.

Other preferred embodiments of the invention will follow from the dependent claims, as well as from the following practical examples.

### EXAMPLE 1

### Micronization of Calcium Peroxide

The micronization is carried out with calcium peroxide which is obtainable as a powder with a content of at least 60% calcium peroxide. Micronization is a dry process that does not require any solvent. In a MC JETMILL® airstream mill (Jetpharma SA) the individual calcium peroxide crystals are accelerated by compressed gas, either nitrogen or air, so that the particles impact against one another and fall apart into smaller and smaller particles. The particle size can be determined using known analytical methods, preferably with a laser device for particle size measurement (Malvern Mastersizer MicroPlus, Size range 0.05-556 micrometers). The particle size that can be achieved by micronization is smaller than 2-3 micrometers for 50% of the particles and smaller than 5-7 micrometers for 90% of the particles.

### EXAMPLE 2

### Toothpaste with Micronized Calcium Peroxide

100 kg of toothpaste is prepared as follows:

| to | | |
|---|---|---|
| Water, germ-free | 51.3657 | kg |
| the following components are added in succession: | | |
| Methylparaben Ph. Eur. | 0.0540 | kg |
| 99.5% glycerol | 10.0000 | kg |
| Nano-lipobelle H-EQ10 (liposomes loaded with 10% | 0.9000 | kg |
| vitamin E and 5% ubiquinone) | | |
| 70% sorbitol | 7.0000 | kg |
| Zinc citrate hydrate | 0.8700 | kg |
| Sodium saccharine | 0.0660 | kg |
| Papain | 0.2000 | kg |
| Bromelain | 0.2000 | kg |
| Phoskadent NA 211 (sodium monofluorophosphate) | 0.8600 | kg |
| Satiagel UTH 18 (carrageenan) | 0.8700 | kg |

This composition is homogenized for at least 30 minutes and is allowed to swell. Then the following are added and incorporated:

| | | |
|---|---|---|
| Prestige sparking green 3518 containing mica, | 0.8000 | kg |
| titanium dioxide (CI77891) and tin oxide | | |
| Martinal ON-310 (aluminum hydroxide) | 3.2600 | kg |
| Sident 8 (silica) | 12.6000 | kg |
| Sident 22 S silicic acid | 7.000 | kg |
| Micronized calcium peroxide | 0.4500 | kg |
| Peppermint oil (natural) | 1.5000 | kg |
| Pistachio green (CI2051 + CI19140) | 0.0043 | kg |
| and finally | | |
| Tego betain L7 | 2.0000 | kg |

aspirated slowly through a funnel, allowed to stand for 2 hours and then homogenized and subjected to vacuum at −1 bar.

### EXAMPLE 3

### Study on the Determination of Tooth Whitening

General Procedure:

In randomly selected subjects, at the beginning as well as after a certain period, suitably after two weeks of the study, the color of the teeth of the upper and lower incisors were determined using a color-measuring apparatus (Spectroshade). During the four weeks before the beginning of the study, no professional tooth cleaning should have been performed and no tooth creams should have been used for eliminating discoloration of teeth.

Course of the Study:

During the initial examination the tooth color is first determined for each subject on the two upper and two lower incisors with Spectroshade. Additionally, the area with the most intensive discoloration is selected. This area is drawn into a tooth drawing in the subject protocol.

The tooth colors measured with the Spectroshade are converted into values of 1 to 16 corresponding to their whiteness using a Vitapan® scale. Thus the lightest tooth color B1 receives the value 1 and the darkest tooth color C4 receives the value 16 (Table I).

TABLE I
Color scale based on the Vitasystem,
arranged according to whiteness

| Color | Scale | |
|---|---|---|
| B1 | 1 | Light |
| A1 | 2 | ↑ |
| B2 | 3 | \| |
| D2 | 4 | \| |
| A2 | 5 | \| |
| C1 | 6 | \| |
| C2 | 7 | \| |
| D4 | 8 | \| |
| A3 | 9 | \| |
| D3 | 10 | \| |
| B3 | 11 | \| |
| A3.5 | 12 | \| |
| B4 | 13 | \| |
| C3 | 14 | \| |
| A4 | 15 | ↓ |
| C4 | 16 | Dark |

The sum of the values of the four measured teeth is divided by four so that an average value is obtained for each patient.

The patients are advised to clean their teeth with the test toothpaste as they are accustomed to do.

After a suitable period of time, usually two weeks, the above examinations are repeated. In addition, the subjects can be asked about a subjective evaluation of the toothpaste regarding the whitening effect using a questionnaire. Appropriately they are also questioned about the safety and tolerability of the toothpaste.

Results from Preliminary Investigations

Thirty subjects completed the clinical study. After 2 weeks, a clear difference in whiteness was observed in comparison to the beginning of the study, both by visual observation as well as with the SPECTROSHADE.

Thus, at the end of the study, that is, after two weeks, a whitening of the teeth was observed by at least 2 and frequently by more than 4 whiteness levels.

In the evaluation of the patient questionnaire, 85% of the participants had the feeling that their teeth were significantly or somewhat whiter. 15% saw no difference. Similarly, a decrease of bad breath was noted, as well as reduced bleeding of the gums.

1. Toothpaste containing one or more micronized non-encapsulated peroxide compounds, whereby 50% of the particles of said one or more peroxide compound have a diameter of less than 3 microns and at least 90% of said particles have a diameter of below 7 microns.

2. Toothpaste according to claim 1 wherein about 50% of said one or more particles have a diameter of less than 2 microns, and at least 90% of said particles have a diameter of below 5 microns.

3. Toothpaste according to claim 1 wherein said peroxide compound is calcium peroxide.

4. Toothpaste according to claim 1 wherein the concentration of said peroxide compound, based on O₂²⁻ is above 0.01%.

5. Toothpaste according to claim 4, said concentration is above 0.05%, preferably between 0.1% and 10%.

6. Toothpaste according to claim 4, said concentration is about 0.1%.

7. Toothpaste according to claim 1 containing one or more enzymes selected from the group consisting of proteases.

8. Toothpaste according to claim 7 containing one or more enzymes selected from the group consisting of cysteine proteases.

9. Toothpaste according to claim 8 containing one or more enzymes selected from the group consisting of papaine and bromelaine.

10. Toothpaste according to claim 1 containing one or more compounds selected from the group consisting of vitamin E and ubiquinone.

11. Method of treating natural or artificial teeth, inlays, dental prothesis comprising the step of applying an effective amount of a toothpaste according to claim 1.
